(19) United States
(12) Patent Application Publication
(10) Pub. No.: US 2022/0370766 A1
(43) Pub. Date: Nov. 24, 2022

(54) WIGGLE CATHETER

(71) Applicant: MANAN SAMIR PANCHOLY, CLARKS SUMMIT, PA (US)

(72) Inventor: MANAN SAMIR PANCHOLY, CLARKS SUMMIT, PA (US)

(21) Appl. No.: 17/882,529

(22) Filed: Aug. 6, 2022

(51) Int. Cl. A61M 25/06; A61M 25/00

(52) U.S. Cl. CPC A61M 25/0662; A61M 25/005; A61M 25/0023; A61M 25/0054; A61M 25/0045; A61M 25/0053; A61M 25/0068; A61M 2025/0681; A61M 2025/0047

(57) ABSTRACT

A wiggle catheter (a dilator) is disclosed to facilitate advancing catheters after puncturing a blood pathway, including without limitation navigation of noncompliant hardware through a diseased lumen. The wiggle catheter includes a proximal section, a distal section, and an intermediate section disposed between the proximal section and the distal section, the intermediate section may include a plurality of angulated kinks along the long axis of the catheter, the angulated kinks having substantially the same diameter at the outer edge of the kinks, said diameter of the outer edge of the kinks being substantially same as the inner diameter of an introducer sheath whereby the wiggle catheter may be positioned inside the introducer sheath with the outer edges of the kinks in close proximity to the inner surface of the introducer sheath. The wiggle catheter may also include a sinusoidal waveform like structure or a helical or spiral structure.

### FIELD

Embodiments described herein concern devices and methods for advancing catheters after puncturing a blood pathway, including without limitation navigation of noncompliant hardware through a diseased lumen.

### BACKGROUND

Endovascular procedures are increasing in numbers, due to increasing ability of current hardware to treat disease conditions that were traditionally treated with open surgery. With growing complexity of disease substrate, as well as growing complexity of hardware to enhance their functionality, difficulties in navigation through diseased lumen is a common problem. Processes such as atherosclerotic degeneration, as well as other conditions such as hypertensive and infiltration processes lead to modifications in the structure of the vessels with changes such as tortuosity, intraluminal deposition of non-conforming material such as plaque or calcium. These changes frequently lead to impediment in the navigation of relatively noncompliant hardware that has to be placed in order to maintain access to the lumen or deliver devices necessary for treatment.

### SUMMARY

An embodiment of the invention comprises a wiggle catheter with a non-linear structure having a series of angulated kinks along the long axis of the wiggle catheter. In another embodiment, the wiggle catheter comprises a sinusoidal waveform like structure. In yet another embodiment, the wiggle catheter comprises a helical or spiral structure.

In one embodiment, the non-linear structural shape of the wiggle catheter may be limited to a frequency of one, e.g., there may be one kink or one wave or one spiral along the axis of the wiggle catheter. In another embodiment, the non-linear structural shape may be repeated along the longitudinal axis of the wiggle catheter with a frequency between 2-30. and may be equidistant or have variable distance between each unit of repetition.

In one embodiment, the shape of each non-linear geometric unit might be identical or substantially similar to the shape of another non-linear geometrical unit along the linear length of the wiggle catheter. In another embodiment, the shape of at least one non-linear geometric unit is different from the shapes of other non-linear geometric units. In yet another embodiment, the shapes of all non-linear geometric units may have different conformation along the linear length of the wiggle catheter.

In one embodiment, the wiggle catheter may have a coating on its exterior that makes it have low friction characteristics. In some embodiments, the coating comprises a hydrophilic material. In yet another embodiment, the wiggle catheter may have a central lumen that accommodates a guide wire.

### BRIEF DESCRIPTION OF DRAWINGS

FIG. 1 is a schematic view of a wiggle catheter having features of one embodiment of the present invention.

FIG. 2 is a schematic view of a wiggle catheter having features of another embodiment of the present invention.

FIG. 3 is a schematic view of a catheter having features of yet another embodiment of the present invention.

FIG. 4 depicts a catheter of an embodiment of the present invention being advanced through a stent in a lumen in a patient's body.

FIG. 5 depicts a catheter of an embodiment of the present invention being advanced through a diseased lumen in a patient's body.

### DETAILED DESCRIPTION

Embodiments described herein provide the user a safe, simple, and reliable device and method to advance a catheter such as an introducer sheath through a tortuous and/or a diseased lumen inside the body of a patient. When the wiggle catheter of the present invention is placed inside an introducer sheath wherein the inner diameter of the introducer sheath is equal or substantially equal to the outer diameter of the wiggle catheter of the present invention, a composite is created. This composite is similar to the composite formed from an introducer sheath and a dilator that are often used in vascular procedures. For ease of reference, the wiggle catheter of the present invention is henceforth called a dilator in this description.

In operation, the composite of an introducer sheath and a dilator of the present invention is loaded over a guidewire, and advanced into a vessel, or any other lumen, and when it encounters either a geometric angle or a turn, or any other non-displaceable geometry, the inner dilator could be turned in a corkscrew fashion or could be simply advanced. In one embodiment of a method of the present invention, the dilator is fixed, and the introducer sheath is advanced over the dilator engaging its undulations and hence “bouncing off” or turning away from the adverse anatomy elements that might obstruct its transit.

A guide wire with a similar structure (Hi-torque Wiggle guidewire, Abbott, USA) with bends in it, works using a similar concept. However, the guidewire is structurally not able to provide the longitudinal stiffness necessary to force the movement of the tip of the introducer sheath or catheter. On the contrary, an opposite motion happens wherein the tip of the introducer sheath or catheter is immobilized, and the guidewire moves around the tip. Due to this lack of longitudinal stiffness, the Wiggle guidewire fails when the anatomy becomes moderate to severely complex or if the hardware being advanced is stiffer than the guidewire.

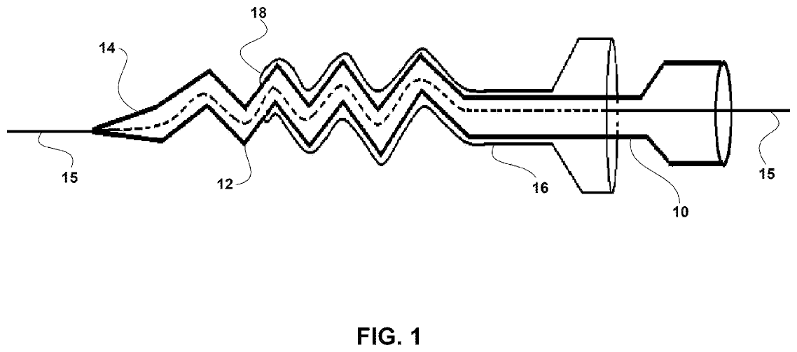

By using a dilator of an embodiment of the present invention (see FIG. 1), with the outer diameter of the dilator 10 same or substantially similar as the inner diameter of the introducer 16, and the protruding length 12 of the dilator having bends or spirals in it which may be angulated, spiral, or in any other geometric conformation, allows for a sturdy longitudinal array over which the tip 18 of the introducer sheath could be advanced. The dilator 10 can be placed inside the introducer sheath 16, with the dilator protruding beyond the distal end 18 of the introducer sheath, and the composite is advanced over the guide wire 15, and when the first resistance point is met, manipulation of the protruding end 14 of the dilator by either a corkscrew or any other form of movement, allows for the navigation of the distal protruding exposed end of the dilator through the adverse anatomy. Once protruding end 14 of the dilator is advanced across the adverse anatomy, the introducer sheath is either advanced over the dilator, or the entire dilator/introducer composite is further advanced through the adverse anatomy. Compared to a guide wire with the bends, the dilator 10 provides a more stable transmission of longitudinal force with more resistance to the flattening of the bands, and hence more displacement of the introducer tip 18 which can then face away from the obstructing element, and successfully navigate the lumen.

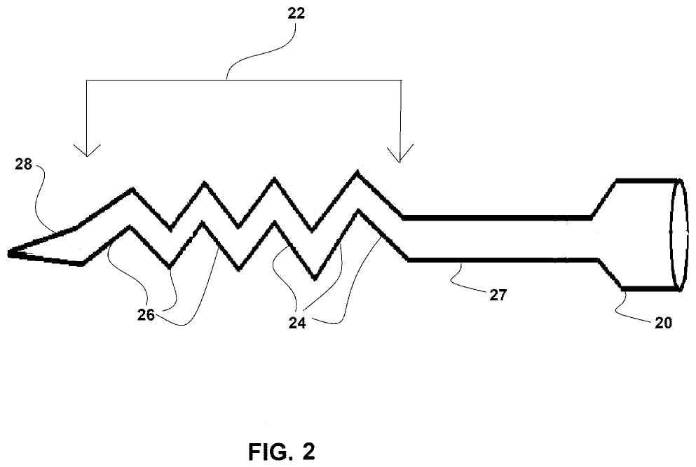

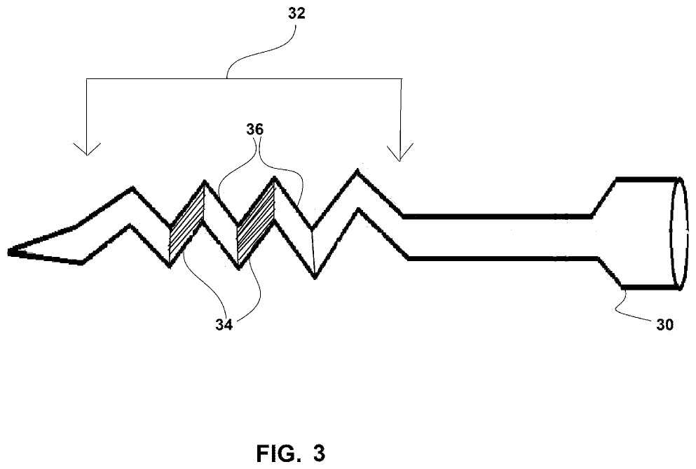

In another embodiment (see FIG. 2), the intermediate section 22 of dilator 20 has a plurality of relatively rigid or stiff segments 24 and a plurality of relatively flexible segments 26. In yet another embodiment of dilator 30 shown in FIG. 3, the stiff segments 34 have a diameter d1, and the relatively flexible segments 36 have a diameter d2 which is smaller than the diameter d1. The stiff segments 34 and relatively flexible segments 36 alternate along a length of the intermediate section 32 and are joined end-to-end along the longitudinal axis of the dilator 30. In another embodiment, the alternating stiff segments 34 and the flexible segments 36 have lengths which typically range from about 0.5 to about 10 cm, preferably about 1 to about 5 cm. The exterior contours of the intermediate segments 34 and 36 smoothly transition between d1 and d2 to provide an undulating shape to the exterior of the intermediate section 32. The intermediate section 32 may be covered with a helical coil or plastic layer (not shown).

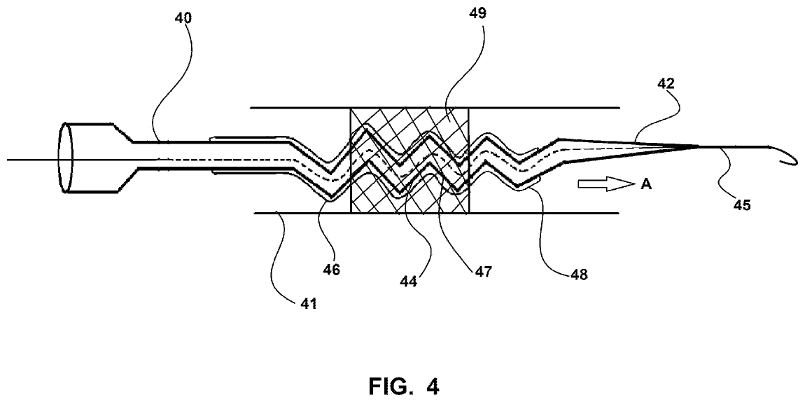

In FIG. 4 the dilator 40 is shown being advanced distally over guidewire 45 in the direction of Arrow A through a stent 49 previously present in a vessel 41. The guidewire 45 is shown extending across the distal end 42 of dilator 40 and out into the body lumen 41. As the dilator 40 is advanced, it conforms by contact to the inner contour of the introducer sheath 46. Note that inner contour of introducer sheath 46 has non-uniform contact with the surface of the intermediate section of dilator 40. The stiff intermediate segments 44 tend to contact the interior surface of introducer sheath 46 while the flexible intermediate segments 47 tend to either not contact the inner surface of the introducer sheath 46 or contact it with less pressure. As a result, the leading distal edge 48 of introducer sheath 46 intermittently curves and straightens laterally as the dilator 40 is advanced.

The construction of an embodiment of the dilator of the present invention is, other than the intermediate section 22 (See FIG. 2), similar to conventional dilators. The overall dilator length may range from about 10 cm to about 330 cm and typically about 170 to 190 cm. The dilator distal segment may include a helical coil, but this may be replaced with a polymer jacket, or the like, which surrounds and covers at least a portion of the distal section 28. The dilator may be of conventional materials, such as stainless steel, high strength precipitation hardened cobalt-chromium or cobalt-chromium-molybdenum alloys such as MP35N, Elgiloy and the like or a super-elastic NiTi alloy. The proximal shaft section 27 may be formed of a material different than the distal segmented shaft. The proximal shaft section 27 of the dilator may have a diameter from about 0.007 to 0.035 inch (0.18-0.89 mm), about 0.01 to about 0.014 inch (0.25-0.36 mm) for coronary uses.

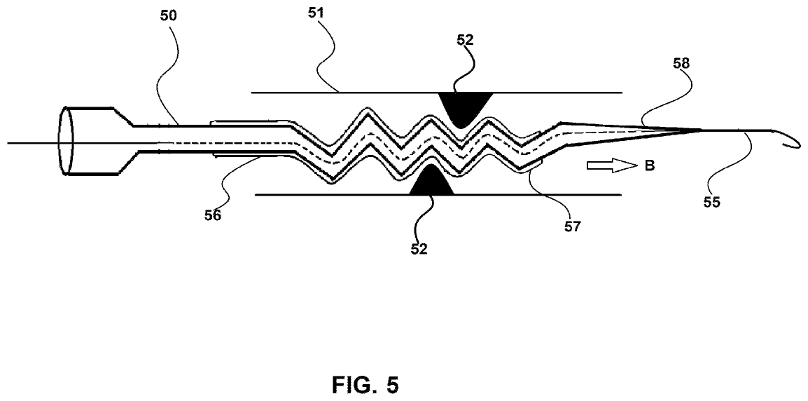

In FIG. 5 the dilator 50 is shown being advanced distally over guidewire 55 in the direction of Arrow B through a diseased lumen 51 in a patient's body. The guidewire 55 is shown extending across the distal end 58 of dilator 50 and out into the body lumen 51. As the dilator 50 is advanced, it conforms by contact to the inner contour of the introducer sheath 56. Note that inner contour of introducer sheath 56 has non-uniform contact with the surface of the intermediate section of dilator 50. The leading distal edge 57 of introducer sheath 56 intermittently curves and straightens laterally, thereby facilitating passing through the obstructive plaque 52 as the dilator 50 is advanced in the diseased lumen 51.

In one embodiment, the dilator (wiggle catheter) of the present invention comprises a proximal section, a distal section, and an intermediate section disposed between the proximal section and the distal section, the intermediate section comprises a plurality of angulated kinks along the long axis of the catheter, the angulated kinks have substantially the same diameter at the outer edge of the kinks, said diameter of the outer edge of the kinks are substantially same as the inner diameter of an introducer sheath whereby the wiggle catheter may be positioned inside the introducer sheath with the outer edges of the kinks in close proximity to the inner surface of the introducer sheath.

In another embodiment, the dilator of the present invention comprises a means for creating alternating relatively stiff angulated kink adjacent relatively flexible angulated kink, and wherein the means for creating relatively stiff angulated kink and relatively flexible angulated kink includes at least one of forming the plurality of angulated kinks of a material which is altered discontinuously along the length and forming the plurality of angulated kinks of different materials having differing mechanical properties.

In yet another embodiment, the means for creating relatively stiff angulated kink and relatively flexible angulated kink further includes alternating cross-sectional areas for the relatively stiff angulated kinks and the relatively flexible angulated kinks.

In yet another embodiment, the means for creating relatively stiff angulated kink and relatively flexible angulated kink further includes alternating lengths of the relatively angulated kinks and the relatively flexible angulated kinks along the long axis of the wiggle catheter.

In yet another embodiment, the means for creating relatively stiff angulated kink and relatively flexible angulated kink further includes alternating the bending rigidity of a material forming the relatively stiff angulated kinks and the relatively flexible angulated kinks.

In yet another embodiment, the means for creating relatively stiff angulated kink and relatively flexible angulated kink further includes intermittently heat-treating along the length of the intermediate section.

In another embodiment, the dilator of the present invention comprises a proximal section, a distal section, and an intermediate section disposed between the proximal section and the distal section, the intermediate section comprises a plurality of sinusoidal waveforms along the long axis of the catheter, the sinusoidal waveforms having substantially the same diameter at the peak of the sinusoidal waveform, said diameter of the peak of the sinusoidal waveform being substantially same as the inner diameter of an introducer sheath whereby the dilator may be positioned inside the introducer sheath with the peaks of the sinusoidal waveforms in close proximity to the inner surface of the introducer sheath.

In another embodiment, the dilator further comprises a means for creating alternating relatively stiff sinusoidal waveform adjacent relatively flexible sinusoidal waveform; and wherein the means for creating relatively stiff sinusoidal waveform and relatively flexible sinusoidal waveform includes at least one of forming the plurality of sinusoidal waveforms of a material which is altered discontinuously along the length and forming the plurality of sinusoidal waveforms of different materials having differing mechanical properties.

In yet another embodiment, the means for creating relatively stiff sinusoidal waveform and relatively flexible sinusoidal waveform includes alternating cross-sectional areas for the relatively stiff sinusoidal waveforms and the relatively flexible sinusoidal waveforms.

In yet another embodiment, the means for creating relatively stiff sinusoidal waveform and relatively flexible sinusoidal waveform includes alternating lengths of the relatively sinusoidal waveforms and the relatively flexible sinusoidal waveforms along the long axis of the wiggle catheter.

In yet another embodiment, the means for creating relatively stiff sinusoidal waveform and relatively flexible sinusoidal waveform includes alternating the bending rigidity of a material forming the relatively stiff sinusoidal waveforms and the relatively flexible sinusoidal waveforms.

In yet another embodiment, the means for creating relatively stiff sinusoidal waveform and relatively flexible sinusoidal waveform includes intermittently heat-treating along the length of the intermediate section.

In another embodiment, the dilator (wiggle catheter) of the present invention comprises a proximal section, a distal section, and an intermediate section disposed between the proximal section and the distal section; the intermediate section comprises a plurality of helical or spiral structures along the long axis of the catheter, the helical or spiral structures having substantially the same diameter at the peak of the helical or spiral structure, said diameter of the peak of the helical or spiral structure being substantially same as the inner diameter of an introducer sheath whereby the dilator may be positioned inside the introducer sheath with the peaks of the helical or spiral structures in close proximity to the inner surface of the introducer sheath.

In yet another embodiment, the dilator comprises a means for creating alternating relatively stiff helical or spiral structure adjacent relatively flexible helical or spiral structure; and wherein the means for creating relatively stiff helical or spiral structure and relatively flexible helical or spiral structure includes at least one of forming the plurality of helical or spiral structures of a material which is altered discontinuously along the length and forming the plurality of helical or spiral structures of different materials having differing mechanical properties.

In yet another embodiment, the means for creating relatively stiff helical or spiral structure and relatively flexible helical or spiral structure includes alternating cross-sectional areas for the relatively stiff helical or spiral structures and the relatively flexible helical or spiral structures.

In yet another embodiment, the means for creating relatively stiff helical or spiral structure and relatively flexible helical or spiral structure includes alternating lengths of the relatively helical or spiral structures and the relatively flexible helical or spiral structures along the long axis of the wiggle catheter.

In yet another embodiment, the means for creating relatively stiff helical or spiral structure and relatively flexible helical or spiral structure includes alternating the bending rigidity of a material forming the relatively stiff helical or spiral structures and the relatively flexible helical or spiral structures.

In yet another embodiment, the means for creating relatively stiff helical or spiral structure and relatively flexible helical or spiral structure includes intermittently heat-treating along the length of the intermediate section.

It will be appreciated that several of the above-disclosed and other features and functions, or alternatives or varieties thereof, may be desirably combined into many other different systems or applications. Also, that various alternatives, modifications, variations, or improvements therein may be subsequently made by those skilled in the art which are also intended to be encompassed by the following claims.

In the description above, for the purposes of explanation, numerous specific requirements and several specific details have been set forth in order to provide a thorough understanding of the embodiments. It will be apparent however, to one skilled in the art, that one or more other embodiments may be practiced without some of these specific details. The particular embodiments described are not provided to limit the invention, but to illustrate it. The scope of the invention is not to be determined by the specific examples provided above. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. In another situation, an inventive aspect may include a combination of embodiments described herein or in a combination of less than all aspects described in a combination of embodiments.

1. A wiggle catheter, comprising: a proximal section, a distal section, and an intermediate section disposed between the proximal section and the distal section; the intermediate section comprising a plurality of angulated kinks along the long axis of the catheter, the angulated kinks having substantially the same diameter at the outer edge of the kinks, said diameter of the outer edge of the kinks being substantially same as the inner diameter of an introducer sheath whereby the wiggle catheter may be positioned inside the introducer sheath with the outer edges of the kinks in close proximity to the inner surface of the introducer sheath.

2. The wiggle catheter of claim 1, further comprising a means for creating alternating relatively stiff angulated kink adjacent relatively flexible angulated kink; and wherein the means for creating relatively stiff angulated kink and relatively flexible angulated kink includes at least one of forming the plurality of angulated kinks of a material which is altered discontinuously along the length and forming the plurality of angulated kinks of different materials having differing mechanical properties.

3. The wiggle catheter of claim 2, wherein the means for creating further includes alternating cross-sectional areas for the relatively stiff angulated kinks and the relatively flexible angulated kinks.

4. The wiggle catheter of claim 2, wherein the means for creating further includes alternating lengths of the relatively angulated kinks and the relatively flexible angulated kinks along the long axis of the wiggle catheter.

5. The wiggle catheter of claim 2, wherein the means for creating further includes alternating the bending rigidity of a material forming the relatively stiff angulated kinks and the relatively flexible angulated kinks.

6. The wiggle catheter of claim 2, wherein the means for creating further includes intermittently heat-treating along the length of the intermediate section.

7. A wiggle catheter, comprising: a proximal section, a distal section, and an intermediate section disposed between the proximal section and the distal section; the intermediate section comprising a plurality of sinusoidal waveforms along the long axis of the catheter, the sinusoidal waveforms having substantially the same diameter at the peak of the sinusoidal waveform, said diameter of the peak of the sinusoidal waveform being substantially same as the inner diameter of an introducer sheath whereby the wiggle catheter may be positioned inside the introducer sheath with the peaks of the sinusoidal waveforms in close proximity to the inner surface of the introducer sheath.

8. The wiggle catheter of claim 1, further comprising a means for creating alternating relatively stiff sinusoidal waveform adjacent relatively flexible sinusoidal waveform; and wherein the means for creating relatively stiff sinusoidal waveform and relatively flexible sinusoidal waveform includes at least one of forming the plurality of sinusoidal waveforms of a material which is altered discontinuously along the length and forming the plurality of sinusoidal waveforms of different materials having differing mechanical properties.

9. The wiggle catheter of claim 2, wherein the means for creating further includes alternating cross-sectional areas for the relatively stiff sinusoidal waveforms and the relatively flexible sinusoidal waveforms.

10. The wiggle catheter of claim 2, wherein the means for creating further includes alternating lengths of the relatively sinusoidal waveforms and the relatively flexible sinusoidal waveforms along the long axis of the wiggle catheter.

11. The wiggle catheter of claim 2, wherein the means for creating further includes alternating the bending rigidity of a material forming the relatively stiff sinusoidal waveforms and the relatively flexible sinusoidal waveforms.

12. The wiggle catheter of claim 2, wherein the means for creating further includes intermittently heat-treating along the length of the intermediate section.

13. A wiggle catheter, comprising: a proximal section, a distal section, and an intermediate section disposed between the proximal section and the distal section; the intermediate section comprising a plurality of helical or spiral structures along the long axis of the catheter, the helical or spiral structures having substantially the same diameter at the peak of the helical or spiral structure, said diameter of the peak of the helical or spiral structure being substantially same as the inner diameter of an introducer sheath whereby the wiggle catheter may be positioned inside the introducer sheath with the peaks of the helical or spiral structures in close proximity to the inner surface of the introducer sheath.

14. The wiggle catheter of claim 1, further comprising a means for creating alternating relatively stiff helical or spiral structure adjacent relatively flexible helical or spiral structure; and wherein the means for creating relatively stiff helical or spiral structure and relatively flexible helical or spiral structure includes at least one of forming the plurality of helical or spiral structures of a material which is altered discontinuously along the length and forming the plurality of helical or spiral structures of different materials having differing mechanical properties.

15. The wiggle catheter of claim 2, wherein the means for creating further includes alternating cross-sectional areas for the relatively stiff helical or spiral structures and the relatively flexible helical or spiral structures.

16. The wiggle catheter of claim 2, wherein the means for creating further includes alternating lengths of the relatively helical or spiral structures and the relatively flexible helical or spiral structures along the long axis of the wiggle catheter.

17. The wiggle catheter of claim 2, wherein the means for creating further includes alternating the bending rigidity of a material forming the relatively stiff helical or spiral structures and the relatively flexible helical or spiral structures.

18. The wiggle catheter of claim 2, wherein the means for creating further includes intermittently heat-treating along the length of the intermediate section.
